(19) United States
(12) Patent Application Publication
(10) Pub. No.: US 2020/0339936 A1
(43) Pub. Date: Oct. 29, 2020

(54) Cell Mechanical Stimulating Device

(71) Applicant: China Medical University, Taichung City (TW)

(72) Inventors: Dan-Jae Lin, Taichung City (TW); Ming-Tzu Tsai, Taichung City (TW); Chao-Wei Hung, Taichung City (TW); Yi-Tao Chang, Taichung City (TW); Chun-Hsu Yao, Taichung City (TW)

(73) Assignee: China Medical University, Taichung City (TW)

(21) Appl. No.: 16/856,203

(22) Filed: Apr. 23, 2020

(30) Foreign Application Priority Data
Apr. 25, 2019 (TW) 108114523

(51) Int. Cl. C12M 1/42; C12M 1/00; C12M 1/34

(52) U.S. Cl. CPC C12M 35/04; C12M 41/40; C12M 23/38

(57) ABSTRACT

A cell mechanical stimulating device for applying to at least one cell sample is provided. The device includes a base unit, at least one moving component and at least one magnetic component. The base unit includes a base body and a separating lid. The base body includes at least one culturing chamber and a bottom chamber surface. The cell sample is disposed in the culturing chamber, and the separating lid encloses the culturing chamber. The magnetic component drives the moving component to shift between a pressurizing position and a non-pressurizing position. At the pressurizing position, the moving component moves toward the bottom chamber surface and pressurizes the cell sample. At the non-pressurizing position, the moving component moves toward the separating lid and the pressure on the cell sample is removed. The cell sample is pressurized by the magnetic component driving the moving component.

### RELATED APPLICATIONS

This application claims priority to Taiwan Application Serial Number 108114523, filed Apr. 25, 2019, which is herein incorporated by reference.

### BACKGROUND

### Technical Field

The present disclosure relates to a biological testing instrument. More particularly, the present disclosure relates to a cell mechanical stimulating device.

### Description of Related Art

A conventional mechanical stimulation cell culture system, as disclosed in Taiwan Patent No. M517743, includes a driving device, a testing device and a loading device adapted for loading a sample under test. The driving device includes a power output unit and a stretching unit, where the stretching unit is driven by the power output unit to reciprocatingly move along a predetermined direction. The testing device includes an outer case and a mechanical stimulating unit detachably arranged at the outer case and detachably connecting between the loading device and the stretching unit. The power output unit includes a power source and a reduction gear set adapted for transmitting the power from the power source to the stretching unit. The stretching unit includes an eccentric component driven by the reduction gear set to eccentrically rotate, a driven component driven by the eccentric component to reciprocatingly move along the predetermined direction, and a connecting shaft connecting between the driven component and the mechanical stimulating unit.

When the conventional mechanical simulation cell culture system is working, the reduction gear set, the eccentric component, the driven component, the connecting shaft and the mechanical stimulating unit are sequentially driven by the power source. The driven component is driven to reciprocatingly move by the eccentric rotation of the eccentric component, and the connecting shaft further drives the mechanical stimulating unit to perform a tensile or compression force on the sample under the stimulating test. In order to prevent any contamination happening and affecting the result, the testing device and the loading device need to be changed in every test to prevent the cell sample under test from being contaminated by the residue of the previous test.

However, the construction and the operation of the conventional mechanical stimulation cell culture system are relatively complicated, which leads to disadvantages of high manufacturing cost and complexity in manipulation.

### SUMMARY

According to one aspect of the present disclosure, a cell mechanical stimulating device for applying to at least one cell sample includes a base unit, at least one moving component and at least one magnetic component. The base unit includes a base body, and a separating lid detachably arranged at the base unit. The base body includes a top surface, a bottom surface opposite to the top surface along a top-bottom-axis, at least one culturing chamber recessed from the top surface and extending to the bottom surface, a bottom chamber surface adjacent to the bottom surface, and an inner periphery surface extending from the bottom chamber surface to the top surface. Each of the at least one culturing chamber is delimited by the bottom chamber surface and the inner periphery surface. The at least one cell sample is respectively disposed in the at least one culturing chamber and arranged at the bottom chamber surface. The separating lid is detachably arranged and abuts the top surface to enclose the at least one culturing chamber. The at least one moving component is top-bottom-axis-movably and respectively arranged in the at least one culturing chamber. Each of the at least one moving component is disposed between the separating lid and the corresponding cell sample. The at least one magnetic component is arranged at the base unit and respectively corresponding to and separated from the at least one culturing chamber. The change of magnetic force of the at least one magnetic component drives the at least one moving component to shift relatively to the at least one cell sample between a pressurizing position and a non-pressurizing position. The at least one moving component moves toward the bottom chamber surface and pressurizes the at least one cell sample when the at least one moving component is at the pressurizing position, and the at least one moving component moves toward the separating lid and the pressure applied on the at least one cell sample is removed when the at least one moving component is at the non-pressurizing position.

### BRIEF DESCRIPTION OF THE DRAWINGS

The present disclosure can be more fully understood by reading the following detailed description of the embodiment, with reference made to the accompanying drawings as follows:

FIG. 1 is a three-dimensional explosive view of a cell mechanical stimulating device according to one example of the present disclosure.

FIG. 2 is a partial cross-sectional view, illustrating a moving component of one example is at a pressurizing position.

FIG. 3 is a partial cross-sectional view, illustrating the moving component of one example is at a non-pressurizing position.

FIG. 4 is a partial cross-sectional view of a cell mechanical stimulating device according to another example of the present disclosure.

FIG. 5 is a partial cross-sectional view, illustrating a moving component of another example is at a non-pressurizing position.

FIG. 6 is a partial cross-sectional view of a cell mechanical stimulating device according to still another example of the present disclosure.

FIG. 7 is a cross-sectional view taken along line 7-7 of FIG. 6.

FIG. 8 is a partial cross-sectional view, illustrating a moving component of still another example is at a pressurizing position.

### DETAILED DESCRIPTION

Before the present disclosure is detailedly described, please note that similar elements are numbered identically in the following description.

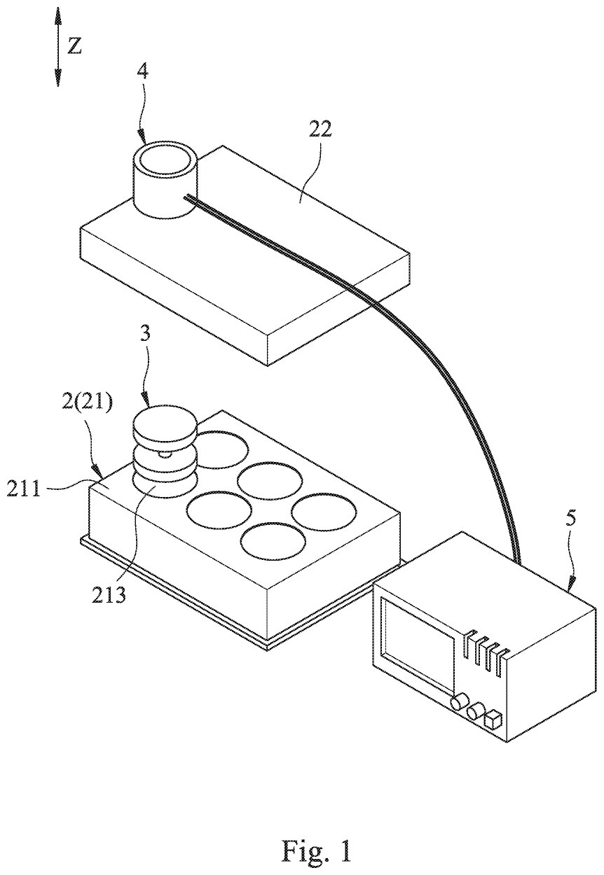

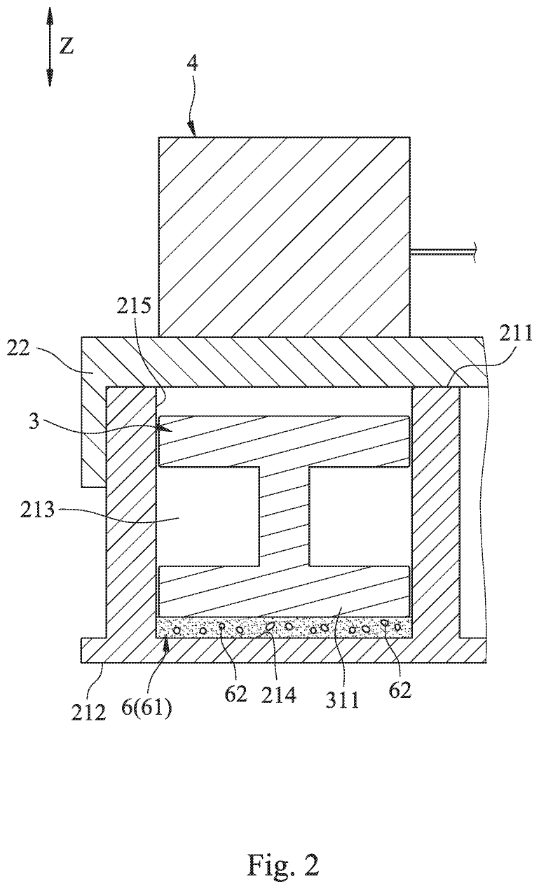

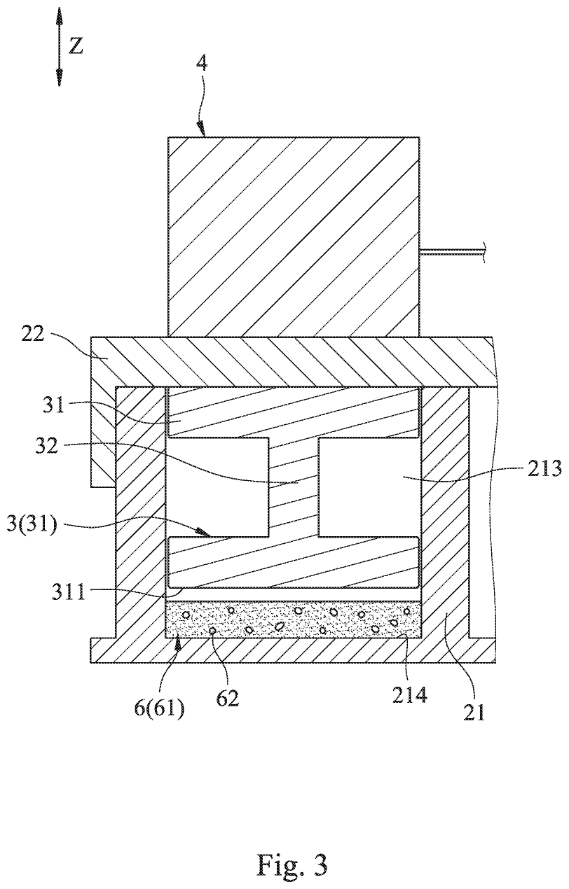

Please refer to FIG. 1, FIG. 2 and FIG. 3. A cell mechanical stimulating device according to one example of the present disclosure is for applying to a plurality of cell samples 6. Each of the cell samples 6 can include a biological carrier 61 and a plurality of cells 62 embedded in the biological carrier 61. The biological carrier 61 can be a protein scaffold, a gel or a sponge. The plurality of cells 62 can be directly cultured in a culture medium without the existence of the biological carrier 61, which is not a limitation to the present disclosure. The features of the biological carrier 61 and the plurality of cells 62 are prior art, and the unnecessary details thereof will not be mentioned herein. The cell mechanical stimulating device includes a base unit 2, a plurality of moving components 3, a plurality of magnetic components 4 and a power supply 5. It should be mentioned that, in this example, the number of the moving components 3, the magnetic components 4 and the cell samples 6 is six, respectively. The number of the moving components 3, the magnetic components 4 and the cell samples 6 can be changed to meet the requirement, which is not a limitation to the present disclosure. It will be illustrated by one moving component 3 and one magnetic component 4 corresponding to one cell sample 6 as the example below.

The base unit 2 includes a base body 21, and a separating lid 22 detachably arranged at the base unit 2. The base body 21 includes a top surface 211, a bottom surface 212 opposite to the top surface 211 along a top-bottom-axis Z, six culturing chambers 213 recessed from the top surface 211 and extending to the bottom surface 212. Each of the culturing chambers 213 is delimited by a bottom chamber surface 214, which is adjacent to the bottom surface 212, and an inner periphery surface 215, which extends from the bottom chamber surface 214 to the top surface 211. Each of the cell samples 6 is disposed in the corresponding culturing chamber 213 and arranged at the bottom chamber surface 214. The separating lid 22 is detachably arranged and abuts the top surface 211 to enclose the culturing chambers 213.

The moving component 3 is top-bottom-axis-movably arranged in the culturing chamber 213. The moving component 3 is disposed between the separating lid 22 and the cell sample 6. The moving component 3 includes two pressing blocks 31 separately arranged along the top-bottom-axis Z, and a shaft 32 connecting between the two pressing blocks 31. The moving component 3 is in an I-shape when observed from the side, and is made of metal materials.

The magnetic component 4 is arranged at the separating lid 22 and corresponding to and separated from the culturing chamber 213. The magnetic component 4 is an electromagnet. It is worth mentioning that, in this example, the magnetic components 4 are respectively corresponding to the moving components 3. In other aspects, the plurality of moving components 3 can be sequentially driven by one magnetic component 4, or be driven by one large magnetic component 4 at the same time, which are not limitations to the present disclosure.

The power supply 5 electrically connects to the magnetic component 4 and is capable of controlling the magnetic force from the magnetic component 4. Specifically, the power supply 5 is capable of controlling the generation, magnitude and frequency of the magnetic force from the magnetic component 4 to apply a desired stimulation on the cell sample 6.

The change of magnetic force of the magnetic component 4 drives the moving component 3 to shift relatively to the cell sample 6 between a pressurizing position (shown in FIG. 2) and a non-pressurizing position (shown in FIG. 3). The moving component 3 moves toward the bottom chamber surface 214 and pressurizes the cell sample 6 when the moving component 3 is at the pressurizing position. The moving component 3 moves toward the separating lid 22 and the pressure applied on the cell sample 6 is removed when the moving component 3 is at the non-pressurizing position.

When the cell mechanical stimulating device is working, the electricity provided by the power supply 5 induces the magnetic force of the magnetic component 4, and the magnetic force attracts the moving component 3 to the non-pressurizing position along the top-bottom-axis Z. Then, the magnetic force of the magnetic component 4 is removed, and the moving component 3 drops to the pressurizing position due to gravity to pressurize the cell sample 6 by the weight thereof. In this regard, the response of the plurality of cells 62 with force applied on can be observed, and the differentiation of the plurality of cells 62 can be controlled according to the pressure applied.

After using the cell mechanical stimulating device, the moving component 3 is reusable after being cleaned and sterilized. The cell sample 6 should be disposed along with the base unit 2 to prevent any contamination happening and affecting the later tests.

Compared to the conventional cell mechanical stimulating device, the moving component 3 is driven by non-contact force according to the present disclosure. Therefore, the structure of the base unit 2 and the moving component 3 is significantly simplified, which reduces the manufacturing cost, leads to a simple manipulation, and reduces the possibility of malfunction.

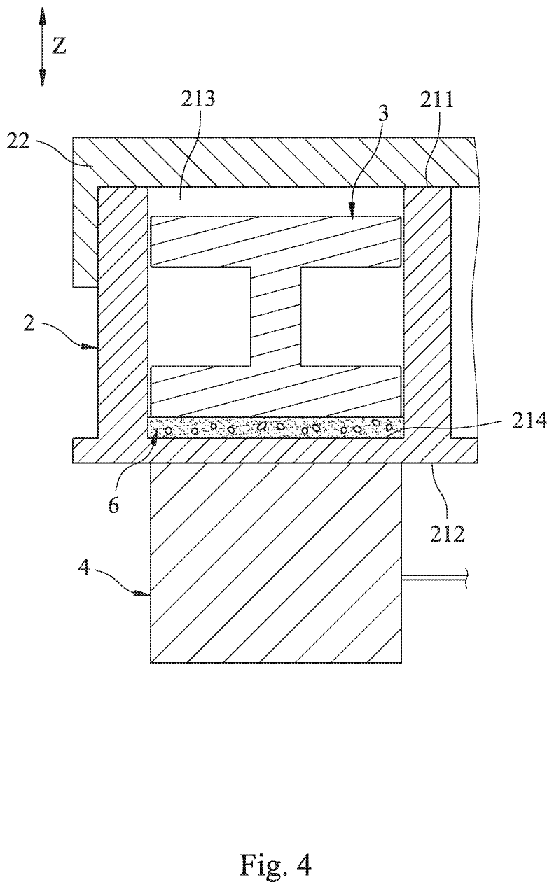

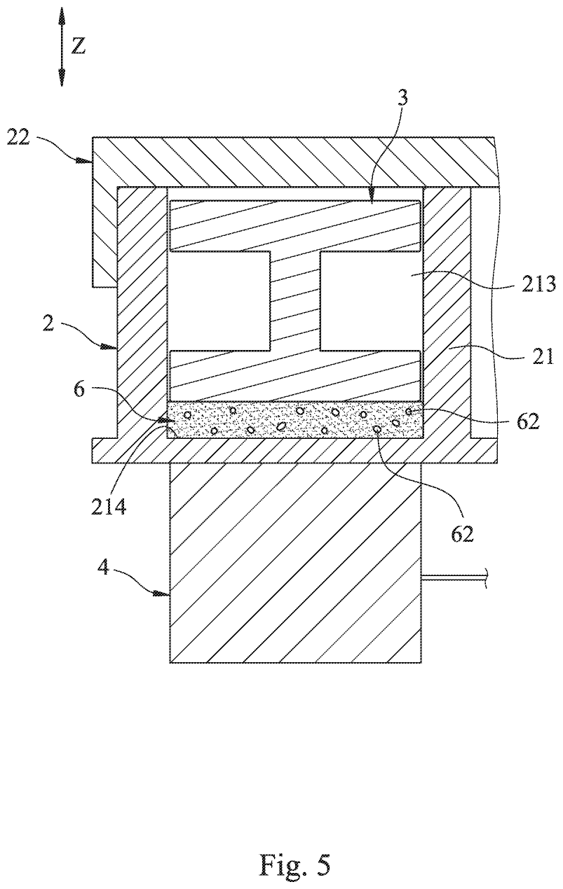

Please refer to FIG. 4 and FIG. 5. Another example of the present disclosure is similar to one example, but has the difference that: the magnetic component 4 is disposed below the base body 21. The magnetic force which the magnetic component 4 applies on the moving component 3 is removed when the moving component 3 is at the pressurizing position, and the moving component 3 is affected by gravity to move toward the bottom chamber surface 214 and pressurizes the cell sample 6 (shown in FIG. 4). The magnetic component 4 repels the moving component 3 by the magnetic force to make the moving component 3 move toward the separating lid 22, and the pressure applied on the cell sample 6 is removed when the moving component 3 is at the non-pressurizing position (shown in FIG. 5).

Therefore, the cell sample 6 is cultured and stimulated by adopting a different force applying method according to another example.

It should be understood that, the position of the moving component 3 in the culturing chamber 213 is changeable according to the magnitude of the magnetic force from the magnetic component 4, and is related to the magnitude of the pressure which the moving component 3 applies on the cell sample 6. Thus, the pressure applied on the cell sample 6 can be adjusted by changing the magnitude of the magnetic force from the magnetic component 4, which is controlled by the power supply 5. Since the electricity provided by the power supply 5 is programmable, the position of the moving component 3 can be programmatically controlled to adjust the pressure applied on the cell sample 6.

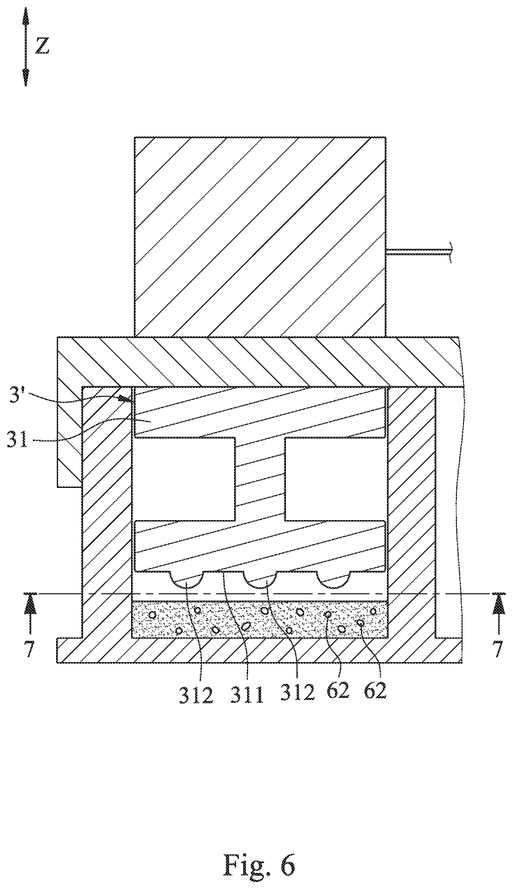

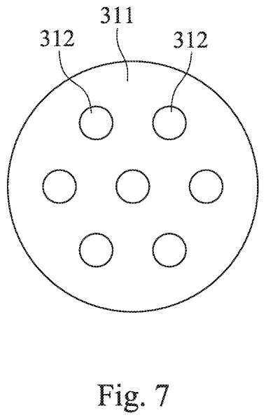

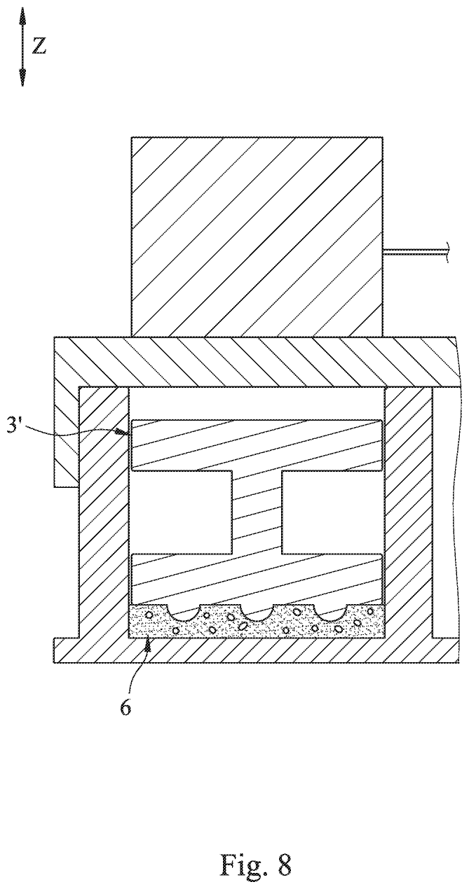

Please refer to FIG. 6, FIG. 7 and FIG. 8. Still another example of the present disclosure is similar to one example, but has the difference that: the pressing block 31 of the moving component 3′ adjacent to the cell sample 6 includes a pressing surface 311 adjacent to the cell sample 6 and a plurality of protruding areas 312 arranged on the pressing surface 311. In this example, the protruding areas 312 are in hemispherical shapes, which is not a limitation to the present disclosure.

Therefore, according to still another example, the cell sample 6 is tested and stimulated by adopting a force applying method which is different from the aforementioned examples.

In summary, in the cell mechanical stimulating device of the present disclosure, the cell sample 6 is pressurized by the magnetic component 4 driving the moving component 3 to shift along the top-bottom-axis Z. Thus, the constructions of the base unit 2 and the moving component 3 are simplified, which reduces the manufacturing cost and leads to a simple manipulation, and the target of the present disclosure is reliably achieved.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A cell mechanical stimulating device for applying to at least one cell sample, comprising:
a base unit, comprising:
a base body, comprising:
a top surface;
a bottom surface opposite to the top surface along a top-bottom-axis;
at least one culturing chamber recessed from the top surface and extending to the bottom surface;
a bottom chamber surface adjacent to the bottom surface, wherein the at least one cell sample is respectively disposed in the at least one culturing chamber and arranged at the bottom chamber surface; and
an inner periphery surface extending from the bottom chamber surface to the top surface, wherein each of the at least one culturing chamber is delimited by the bottom chamber surface and the inner periphery surface;
a separating lid detachably arranged at the base unit, wherein the separating lid is detachably arranged and abuts the top surface to enclose the at least one culturing chamber;
at least one moving component top-bottom-axis-movably and respectively arranged in the at least one culturing chamber, wherein each of the at least one moving component is disposed between the separating lid and the corresponding cell sample; and
at least one magnetic component arranged at the base unit and respectively corresponding to and separated from the at least one culturing chamber, wherein the change of a magnetic force of the at least one magnetic component drives the at least one moving component to shift relatively to the at least one cell sample between a pressurizing position and a non-pressurizing position;
wherein the at least one moving component moves toward the bottom chamber surface and pressurizes the at least one cell sample when the at least one moving component is at the pressurizing position, and the at least one moving component moves toward the separating lid and the pressure applied on the at least one cell sample is removed when the at least one moving component is at the non-pressurizing position.

2. The cell mechanical stimulating device of claim 1, wherein the at least one magnetic component is disposed above the separating lid;
wherein the magnetic force which the at least one magnetic component applies on the at least one moving component is removed when the at least one moving component is at the pressurizing position, and the at least one moving component is affected by gravity to move toward the bottom chamber surface and respectively pressurizes the at least one cell sample; and
wherein the at least one magnetic component attracts the at least one moving component by the magnetic force to make the at least one moving component move toward the separating lid, and the pressure applied on the at least one cell sample is removed when the at least one moving component is at the non-pressurizing position.

3. The cell mechanical stimulating device of claim 2, wherein a position of the at least one moving component in the at least one culturing chamber is changeable according to a magnitude of the magnetic force of the at least one magnetic component.

4. The cell mechanical stimulating device of claim 1, wherein the at least one magnetic component is disposed below the base body;
wherein the magnetic force which the at least one magnetic component applies on the at least one moving component is removed when the at least one moving component is at the pressurizing position, and the at least one moving component is affected by gravity to move toward the bottom chamber surface and respectively pressurizes the at least one cell sample; and
wherein the at least one magnetic component repels the at least one moving component by the magnetic force to make the at least one moving component move toward the separating lid, and the pressure applied on the at least one cell sample is removed when the at least one moving component is at the non-pressurizing position.

5. The cell mechanical stimulating device of claim 4, wherein a position of the at least one moving component in the at least one culturing chamber is changeable according to a magnitude of the magnetic force of the at least one magnetic component.

6. The cell mechanical stimulating device of claim 1, wherein each of the at least one moving component comprises two pressing blocks separately arranged along the top-bottom-axis, and a shaft connecting between the two pressing blocks.

7. The cell mechanical stimulating device of claim 6, wherein one of the two pressing blocks of the moving component adjacent to the corresponding cell sample comprises a pressing surface adjacent to the corresponding cell sample and a plurality of protruding areas arranged on the pressing surface.

8. The cell mechanical stimulating device of claim 1, wherein each of the at least one magnetic component is an electromagnet, and the cell mechanical stimulating device further comprises a power supply electrically connecting to the at least one magnetic component and capable of controlling the magnetic force from the at least one magnetic component.
